(19) United States
(12) Patent Application Publication
(10) Pub. No.: US 2021/0217503 A1
(43) Pub. Date: Jul. 15, 2021

(54) INFORMATION CONFLICT IDENTIFICATION AND CHARACTERIZATION IN RESPONSE TO DATA CARRY-OVER TRANSITIONS

(71) Applicant: KONINKLIJKE PHILIPS N.V., Eindhoven (NL)

(72) Inventors: LUCAS DE MELO OLIVEIRA, CAMBRIDGE, MA (US); GABRIEL RYAN MANKOVICH, BOSTON, MA (US)

(21) Appl. No.: 16/740,568

(22) Filed: Jan. 13, 2020

(51) Int. Cl. G16H 15/00; G16H 10/60; G16H 70/00; G06N 5/04; G06N 20/00; G06F 40/40

(52) U.S. Cl. CPC G16H 15/00; G16H 10/60; G06F 40/40; G06N 5/04; G06N 20/00; G16H 70/00

(57) ABSTRACT

Implementations described herein relate to processing concept-based conflicts, between different instances of clinical data, in response to a user initializing a transfer of previously existing clinical data into a current report. The current report can thereafter be referenced by another user who, as a result of the concept-based conflict check, will be put on notice of conflict(s) between instances of clinical data. Conflicts can be based on criterion corresponding to particular clinical concepts, and a clinical concept can be identified using clinical data that is the subject of transfer to a current report. For instance, natural language processing can be performed on clinical data to identify clinical concepts characterized by the clinical data. In some implementations, criterion can be generated for a clinical concept based on one or more trained machine learning models, which can be trained using medical reference data and/or patient medical records.

### TECHNICAL FIELD

Implementations set forth herein relate to generating conflict-related data when transferring medical data between data sources, and rendering the conflict-related data at an interface to subsequently streamline clinical pathways for users referencing the interface.

### BACKGROUND

Medical professionals may rely on a variety of different sources when generating a report concerning, for example, a diagnosis of a patient. Because of the frequency at which the medical professional is requested to generate such reports, the professional may rely on quick copy operations in order to transfer data between different sources. For instance, a medical professional that is generating a report, in response to a patient receiving an MRI, may seek existing data about a disease that is affecting the patient. In order to retrieve such data, the medical professional may access historical data, such as medical notes from a hospital database generated when the patient was once admitted to a hospital. However, if more accurate data related to the disease has been available since the patient was admitted to the hospital, and the medical professional does not identify the more accurate data, the report they generate may not be accurate. These inaccuracies can result from contradictory statements being made about a disease, such as available clinical pathways for treating the disease. As a result, resources expended on treating a disease may be wasted, should any other medical professionals rely on the inaccurate report for identifying and employing certain treatments (e.g., prescribing medicines, arranging a medical procedure, ordering a lab test, etc.).

### SUMMARY

Implementations described herein relate to processing concept-based conflicts, between different instances of clinical data, in response to a user initializing a transfer of previously existing clinical data into a current report. An instance of clinical data can correspond to a clinical concept, and the clinical concept can be affected by various criterion over time. A process for resolving conflicts between different sources of clinical data can be employed to ensure that any clinical data copied into newly generated reports (i.e., sources) satisfy all criterion for each clinical concept characterized by each respective source of clinical data.

A criterion for a clinical concept can refer to a restriction on how the clinical concept can be characterized by clinical data (e.g., medical notes, medical records, lab reports, etc.). An example of a criterion can be a restriction, which provides that a “chronic” illness cannot be characterized as “acute” without additional information describing a context of the “chronic” and/or the “acute” descriptor. Another example of a criterion can be a restriction, which provides that a “benign” tumor cannot be characterized as a “malignant” tumor without additional information describing the context (e.g., dates and/or times of such characterizations) of the “benign” and/or the “malignant” descriptor. In some implementations, additional information can be generated in response to one or more processors determining that a conflict between clinical data renders a criterion unfulfilled. The additional information can then be presented to the user and/or incorporated into a source that is an end target for a transfer of clinical data.

In some implementations, a user can be operating an application via a user interface of a computing device that provides access to various sources of clinical data. The user can identify a first report from which the user would like to copy data into a target report (e.g., a report being newly generated). The first report can include natural language content characterizing a clinical concept, such as a congenital autoimmune disease. Furthermore, the application can characterize a criterion corresponding to the clinical concept, and the criterion can be based on one or more trained machine learning models, one or more statistical models, one or more heuristic formulations, one or more neuro-linguistic program (NLP) processes, and/or any other data from which a criterion can be characterized. As an example, a criterion for a clinical concept, such as a “congenital autoimmune disease,” can be generated to ensure that a newly created report characterizing the clinical concept does not also provide natural language content associated with terms such as “acute.”

For instance, in a situation in which a lab technician is assisting a patient by generating a lab report for a primary care physician, the lab technician can copy data from another lab report that describes the patient as having an “acute disease.” In response to the lab technician initializing the copying of data, another source of data, such as hospital medical records, can be cross-referenced in order to determine whether any conflicts exist relative to the other lab report. If the hospital records include content describing the patient as having a “congenital autoimmune disease,” then data characterizing this apparent conflict can be generated and incorporated into the lab report being generated. In this way, the lab technician, and/or the primary care physician who will later rely on the report, can acknowledge the apparent conflict, and/or at least better understand the history of the patient. This can streamline clinical pathways for patients, thereby eliminating a wasting of resources that might otherwise occur when a medical professional relies on inaccurate data. As an example, in the aforementioned instance, another medical professional might otherwise rely on the lab report characterizing the patient as having an “acute” disease, rather than a “congenital autoimmune disease,” and select an inappropriate clinical pathway for the patient as a result. Should that clinical pathway require medications and/or procedures that have been proven to be ineffective for someone with a “congenital autoimmune disease,” resources spent providing those medications and/or procedures to the patient would be wasted.

In some implementations, a method implemented by one or more processors is set forth as including operations such as processing, via the one or more processors and in response to an input received at a user interface of a computing device, input data that indicates a user has initialized a transfer of first data from a first source to a target source. The operations can further include identifying, based on processing the input data, (i) first data characterizing a clinical feature of one or more patients, and (ii) a criterion corresponding to the clinical feature, wherein the first data is based on natural language content of the first source. The operations can further include identifying, based on identifying the first data, a second source that is different from the first source and associated with the clinical feature, wherein the second source includes other natural language content that is associated with the clinical feature. The operations can further include determining whether the first data and the second data satisfy the criterion corresponding to the clinical feature. The operations can further include, when the first data and the second data are determined to not satisfy the criterion corresponding to the clinical feature: generating supplement data that indicates the first data and the second data do not satisfy the criterion corresponding to the clinical feature, and causing, at least in response to the input received at the user interface of the computing device, the user interface, or another user interface of another computing, to graphically render the first data and the supplemental data.

In some implementations, the target source is an application file generated by the user via the user interface of the computing device, and the application file includes a field to which the first data is to be incorporated. In some implementations, causing the user interface, or the other user interface of the other computing device, to render the first data and the supplemental data includes: causing the user interface, or the other user interface of the other computing device, to simultaneously render the first data and the supplemental data. In some implementations, determining whether the first data and the second data satisfy the criterion includes determining whether a temporal aspect of the first data and another temporal aspect of the second data satisfy the criterion. In some implementations, the temporal aspect characterizes a time at which the first data was generated or last modified, and the other temporal aspect characterizes another time at which the second data was generated or last modified. In some implementations, wherein the first data is provided by the first source, and wherein determining whether the first data and the second data satisfy the criterion includes determining whether the second data characterizes conflicting content relative to the clinical feature characterized by the first data. In some implementations, the criterion is generated based on one or more trained machined learning models that are trained according to patient medical records and/or medical reference data.

The above description is provided as an overview of some implementations of the present disclosure. Further description of those implementations, and other implementations, are described in more detail below.

Other implementations may include a non-transitory computer readable storage medium storing instructions executable by one or more processors (e.g., central processing unit(s) (CPU(s)), graphics processing unit(s) (GPU(s)), and/or tensor processing unit(s) (TPU(s)) to perform a method such as one or more of the methods described above and/or elsewhere herein. Yet other implementations may include a system of one or more computers and/or one or more robots that include one or more processors operable to execute stored instructions to perform a method such as one or more of the methods described above and/or elsewhere herein.

The term “network” as used herein refers to any interconnection of two or more devices (including controllers or processors) that facilitates the transport of information (e.g., for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

The term “user interface” as used herein refers to an interface between a human user or operator and one or more devices that enables communication between the user and the device(s). Examples of user interfaces that may be employed in various implementations of the present disclosure include, but are not limited to, switches, potentiometers, buttons, dials, sliders, a mouse, keyboard, keypad, various types of game controllers (e.g., joysticks), track balls, display screens, various types of graphical user interfaces (GUIs), touch screens, microphones and other types of sensors that may receive some form of human-generated stimulus and generate a signal in response thereto.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 illustrates a view of how a system for conflict checking carry-over data can be employed using one or more criterion corresponding to one or more clinical concepts.

FIG. 2 illustrates a system for detecting clinical report outliers using context-sensitive carryover transitions.

FIG. 3A and FIG. 3B illustrate methods for identifying conflicts between clinical concepts described in different sources of information, and providing supplemental data when problematic data is subject to carry over into a newly generated report.

FIG. 4 is a block diagram of an example computer system.

### DETAILED DESCRIPTION

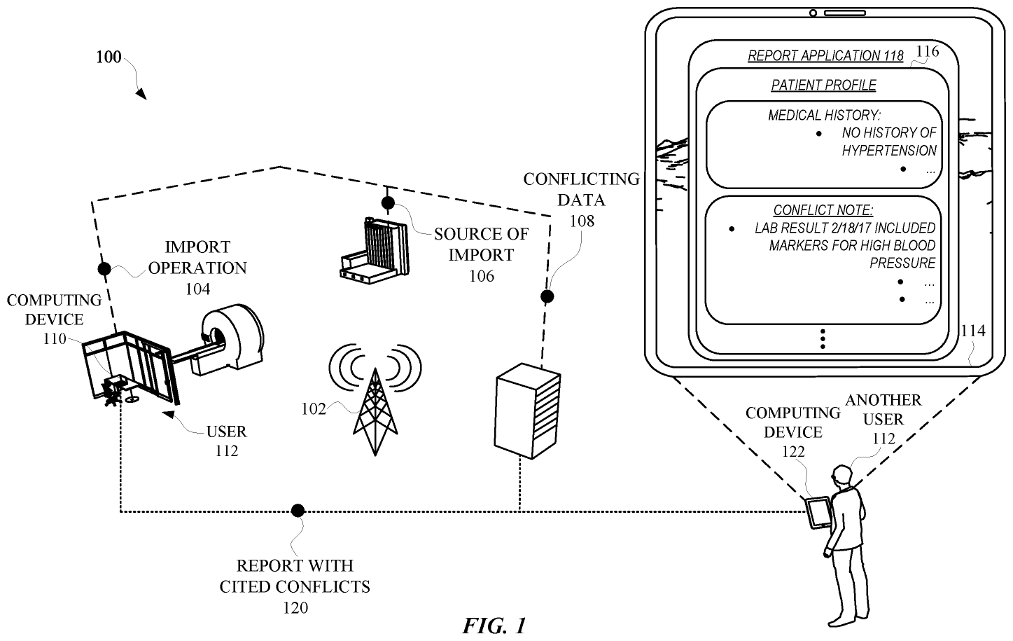

FIG. 1 illustrates a view 100 of how a system for conflict checking carry-over data can be employed using one or more criterion corresponding to one or more clinical concepts. Specifically, conflict check for carry-over data can be performed when an import operation 104 is initialized by a user 112, such as a medical professional tasked with performing a test on a patient (e.g., on MRI scan of a patient). A patient can undergo the test and the user 112 can be responsible for generating a report corresponding to the test. As part of the report, the user 112 may need provide information into various fields of a report, such as medical history of the user, current medications, notes from previous testing, existing diseases, and/or any other relevant medical information. In order to streamline this provisioning of data into the report, the user 112 can initialize the import operation 104, in order to carryover existing data from a source of import 106, such as a hospital database, over a network 102, such as the internet. However, without any conflict-checking system, the user 112 may not realize the data, which they are importing for a patient, conflicts with other existing data that is available from another source. Therefore, by employing a system for conflict checking, at least in response a carry-over data operation being initialized, clinical pathways for treatment can be more readily identified. This can reduce waste of resources that would otherwise occur when inconsequential clinical pathways are selected based on inaccurate data.

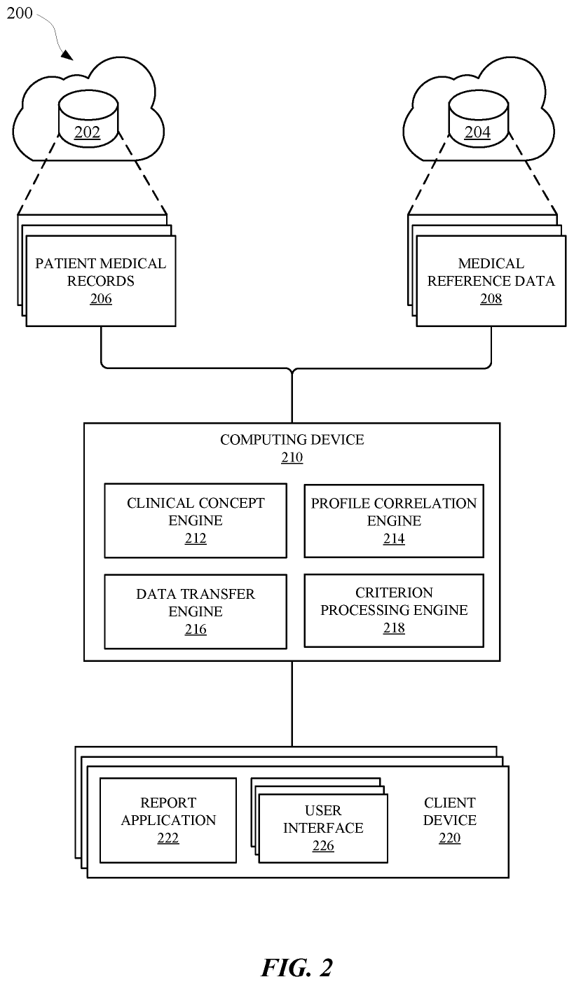

As an example, the user 112 can cause medical history information to be imported from the source of import 106, to a particular field of a report being newly generated by the user 112. In response to the user 112 initializing the import operation 104, a computing device 110, and/or another device that is accessible to the computing device 110, can initialize one or more conflict checks with respect to the medical history information. In some implementations, the conflict check can be performed by an application at the computing device 110, which can identify conflicting data 108 that is stored at a separate device (e.g., a server). For instance, the computing device 110 can include the features of computing device 210 discussed with respect to FIG. 2. Additionally, or alternatively, the computing device 110 can include the features of client device 220, but can access the computing device 210 in order to cause conflict checking of carry-over data.

In some implementations, in order to identify data that may be in conflict with the data being carried over into a report, the data being imported, and/or data that is already identified by the report, can be processed to determine whether any of the data corresponds to a clinical concept. When the data corresponds to one or more clinical concepts, the computing device 110 can identify one or more criterion corresponding to each clinical concept of the one or more clinical concepts. A criterion for a particular clinical concept can be used to determine whether a conflict with the data exists, at least based on whether any data associated with the patient and the clinical concept are in conflict. Furthermore, there can be one or more different criterion that can be unfulfilled for multiple different reasons. For example, a temporal aspect of data being imported, and another temporal aspect of existing data, can fail to satisfy a criterion because the temporal aspects of the data do not correspond to a particular temporal order, do not satisfy a period threshold, and/or are otherwise in conflict. In some implementation, a temporal aspect of data can refer to when the data was generated, when the data was last modified, when the data was last accessed, and/or any other temporal aspect that can be associated with data. Alternatively, or additionally, a criterion can be unfulfilled when natural language content of data being imported substantively conflicts with existing data.

As an example, the user 112 can attempt to carry over existing data from a source of import 106, and some of the data can include natural language content such as “no history of hypertension.” The user 112 can be attempting to import the existing data into a patient profile that includes a field such as, “medical history.” In response to the import operation 104, a conflict check can be initialized in order to determine whether any other existing data conflicts with the natural language content: “no history of hypertension.” In some implementations, the natural language content can be processed to identify whether the natural language content refers to a clinical concept (i.e., a clinical feature). When a clinical concept is identified, one or more criterion associated with the clinical concept can also be identified. For example, a criterion associated with the clinical concept “no hypertension,” can indicate that a conflict exists when there is at least one report, issued previous in time to the data being imported, which provides an indication and/or description of high blood pressure symptoms. For example, a conflict can be established from this criterion when conflicting data 108 characterizes a lab result which was issued before a time when the data “no history of hypertension” was generated, and included data characterizing markers for high blood pressure.

In response to identifying the conflict between the data being carried over and the conflicting data 108, a report application 118 can incorporate the targeted data into a targeted field of the report (e.g., the “medical history” field) and a note characterizing the conflict can be automatically populated into the same report. For example, a graphical interface 116 of the report application 118 can be generated in response to the criterion not being satisfied, and can simultaneously provide the targeted data that was the target of the carryover as well as a description of the data that conflicted with the clinical concept characterized by the targeted data. In this way, another user 112, such as a primary care physician, can thereafter access the report generated by the user 112 via another computing device 122. The other user 112 can then access the report application 118 via a display interface 114 of the computing device 122, and can be put on notice of the conflict between the medical history data (e.g., from the hospital database) and the lab results (e.g., from a server associated with a service provider).

FIG. 2 illustrates a system 200 for detecting clinical report outliers using context-sensitive carryover transitions. In order to generate notes and/or other information within a report, a medical professional and/or other user of the system 200, can access existing data from which to copy and/or otherwise transition into a report that the user is working on. Although the user may find convenience in copying from existing sources, such copying without conflict checks can result in inaccurate and/or contradictory statements being incorporated into a targeted report. However, the system 200 allows for adaptive conflict checking over time, as a user relies on various different sources for information when creating reports.

The system 200 can include one or more client devices 220 through which one of more users can access various sources of data and/or generate reports from the data. For example, a client device 220 can include a report application 222 which the user can access data such as patient medical records 206 and/or medical reference data 208. The report application 222 can access a computing device 210, such as a server device, over a network when accessing a report for a particular patient and/or generating a report for a particular patient. A report can include one or more different fields that include information provided from different databases, such as a first database 202, which can include the patient medical records 206, and/or a second database 204, which can include the medical reference data 208.

In some implementations, a report for a particular patient can include one or more embedded links, which can be generated by a profile correlation engine 214 of the computing device 210, and provide addresses for sources of data populating a report, such as a patient profile. In some implementations, a patient profile that is accessible via the report application 222 can provide a timeline of patient data for a particular patient. The patient data can be sourced from different sources, such as the patient medical records 206 and the medical reference data 208. Furthermore, a user can make changes to a patient profile by, for example, copying information from a patient medical record 206 into the patient profile. The system 200 can ensure that the information being transferred into the patient profile does not conflict with patient data that is already provided in the patient profile.

When the user initializes a transfer of data at the user interface 226 of a report application 222, a data transfer engine 216 of the computing device 210 can detect that the transfer of data has been initialized. In response, the data transfer engine can identify the data that the user is intending to copy into the patient profile. Furthermore, the data transfer engine 216 can identify other data that is associated with the data that the user is intending to copy into the patient profile. The data and/or the other data can be provided, by the data transfer engine 216, to a clinical concept engine 212 of the computing device 210. Alternatively, or additionally, the data transfer engine 216 can provide one or more links to the data and the other data to the clinical concept engine 212 for further processing.

The clinical concept engine 212 can access the data and/or the other data in order to determine whether any of the data refers to one or more clinical concepts. For example, the user may be intending to incorporate historical data about a patient, such as a patient medical record 206 that indicates the user has “no history of hypertension.” The clinical concept engine 212 can determine, using the medical reference data 208, that the portion of data that the user is intending to copy correlates to at least a clinical concept such as “high blood pressure.” Based on this determination that the historical data to be copied (e.g., “no history of hypertension) correlates to the clinical concept “high blood pressure,” the clinical concept engine 212 can communicate the identified clinical concept (e.g., high blood pressure) to a criterion processing engine 218 of a computing device 210.

The criterion processing engine 218 can identify one or more different criterion that influence one or more respective clinical concepts. For example, a clinical concept can be influenced by a criterion, such as a temporal placement of the clinical concept with respect to other clinical concepts that have been correlated to the patient over time. In some implementations, the criterion processing engine 218 can determine that an indication of “no hypertension” cannot be incorporated into a report subsequent to any other record for the patient indicating that the patient has high blood pressure, at least not without other supplemental data being incorporated in order to explain the apparent contradiction. One or more criterion used by the criterion processing engine 218 can be based on one or more neuro-linguistic program (NLP) processes, and/or one or more trained machine learning models, which can optionally be trained using the medical reference data 208 and/or patient medical records 206, which can correspond to one or more different patients. When the criterion processing engine 218 determines that a criterion for a particular clinical concept is not satisfied, the criterion processing engine can generate supplemental data, which can be supplanted into the target report that the user is intending to copy data into.

By leveraging one or more processing devices in order to establish multiple criterion through which to conflict check data transfer processes, medical professionals and/or any other professional can eliminate error generation that would otherwise occur if the automatic criterion checking was not available. However, according to the system 200, conflict checking between criterion generated from patient medical records 206 and medical reference data 208 can be employed automatically in response to the data transfer engine 216 detecting that a user is attempting to copy existing data into the targeted report.

In some implementations, information in a patient profile can be linked to a variety of different sources by a profile correlation engine 214. The profile correlation engine can manage links between a patient profile, which can be accessible via the report application 222, and patient medical records 206 and/or medical reference data 208. For example, an excerpt from a patient medical record 206 can state that the user currently has high blood pressure. The term “high blood pressure” can be correlated to information in the medical reference data 208 by the profile correlation engine 214. This correlation can be embodied as a link that is accessible to the report application 222, and the information from the medical reference data 208 can include natural language content such as “hypertension.” In this way, in response to the user creating a brand new report and initializing a transfer of data from another patient medical record 206, which states “no history of hypertension,” the term “hypertension” and any other correlated terms can be used as search terms to identify related to patient medical records 206. Because the term “hypertension” was correlated to the term “high blood pressure” in the excerpt from the patient medical record 206, the term high blood pressure and/or hypertension can be used when generating supplemental data, should at least one criterion associated with high blood pressure and/or hypertension not be satisfied.

In some implementations, the profile correlation engine 214 can process patient medical records 206 that are being created continually by various different third-party sources. A third party source can be one that is different from a source that provided the support application, the client device 220, the computing device 210, and/or the criterion processing engine 218. Therefore, if a particular patient undergoes a lab test, such as genetic testing, results of the testing can be indicated in a patient medical record 206 and processed, with permission from the patient, by the profile correlation engine 214. The profile correlation engine 214 can search for correlations between terms in the lab results report and the medical reference data 208. When the profile correlation engine 214 identifies a correlation between a term in a lab result and a clinical concept, using the medical reference data 208, the profile correlation engine 214 can generate a link mapping the lab results to a particular clinical concept identified using the medical reference data 208. Thereafter, when a medical professional is generating a patient profile for a subsequent test or procedure, and the medical professional attempts to copy terms corresponding to the particular clinical concept, when one or more criterion corresponding to the particular clinical concept can be processed to determine whether any existing patient medical records 206 fail to fulfill the one or more criterion.

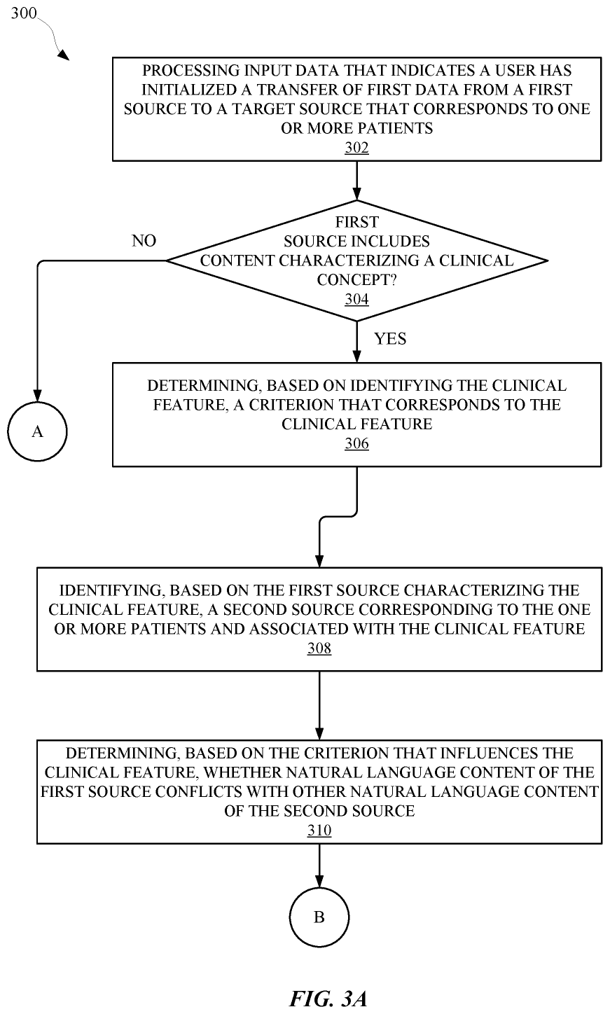

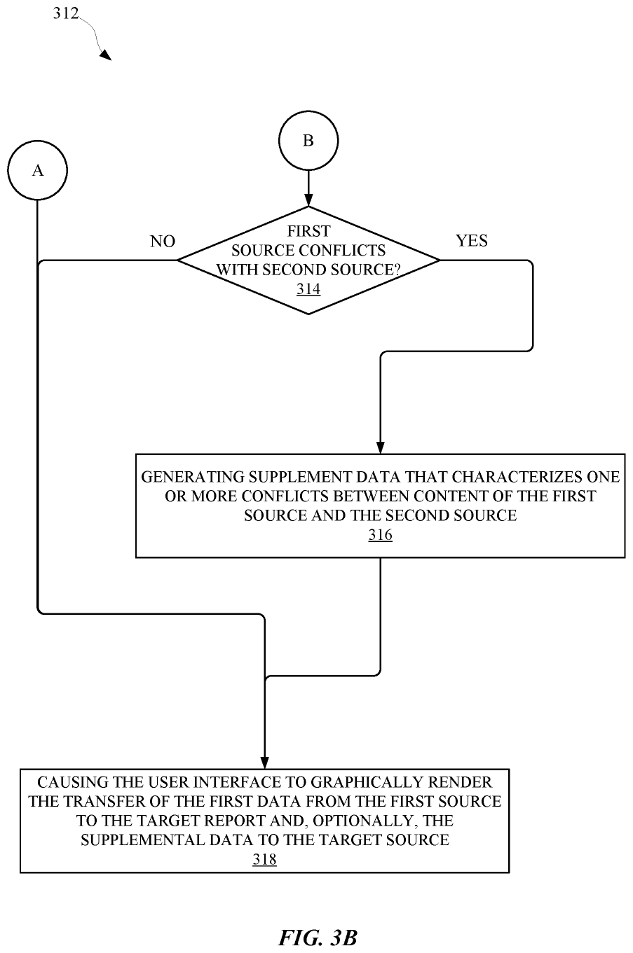

FIG. 3A and FIG. 3B illustrate a method 300 and a method in 312, respectively, for identifying conflicts between clinical concepts described in different sources of information, and providing supplemental data when problematic data is subject to carry over into a newly generated report. The method 300 and the method 312 can be performed by one or more computing devices, applications, and/or any other apparatus or module capable of transferring copies of data. The method 300 can include an operation of processing input data that indicates a user has initialized a transfer of first data from a first report to a target report that corresponds to one of more patients. The user can be, for example, a medical professional that is interacting with a user interface of a computing device. The user may wish to generate the target report in order to characterize an operation recently performed on one or more patients. However, in order to efficiently generate the target report, the user can access other sources of data from which the user can copy existing data. For example, the target report can have a field for natural language content to be provided for describing any existing illnesses of a patient. In order to identify existing data describing the existing illnesses, the user can access a first report provided by a first source, such as a database of hospital records managed by a hospital.

The method of 300 can further include an operation 304 of determining whether the first report includes content characterizing one or more clinical concepts. A clinical concept can be, but is not limited to, a medical condition, medical treatment, and/or any other concept that can be the subject of, or the product of, clinical analysis. For example, in furtherance of the aforementioned example, the first report can characterize a disease of the patient such as acute intestinal pain. In some implementations, determining whether the first report includes content characterizing a clinical concept can include generating a summary of at least a portion of the content of the first report. The summary can then be further processed to determine whether the summary characterizes, or is otherwise associated with, one or more clinical concepts. As an example, data characterizing natural language content of the first report can be applied to one or more trained machine learning models, which can be used to generate the summary of the first report. Furthermore, other data characterizing content of the summary can be applied to one or more other trained machine learning models, which can be used to identify one or more clinical concepts that the first report describes, and/or is otherwise associated with.

When the first report does not include content characterizing a clinical concept (e.g., when the first report exclusively includes data, such as an address, that is presumably inconsequential, at least from a medical standpoint), the method 300 can proceed via continuation element “A.” Specifically, the method 300 can proceed from the operation 304 to the operation 318 at the method 312, at least when the first report does not include content characterizing a clinical concept. However, if at the operation 304 the first report is determined to included content characterizing one or more clinical concepts (i.e., one or more clinical features), the method 300 can proceed to the operation 306.

The operation 306 can include determining, based on identifying the clinical feature, a criterion that influences the clinical feature. A criterion that influences the clinical feature can refer to, but is not limited to, one or more rules manually established by one or more users, and/or one or more rules generated by one or more trained machine learning models. For example, one or more machine learning models can be trained using existing medical data in order to generate rules from patterns that are apparent for certain clinical data. For example, a learned rule can be one that does not allow a chronic disease to be later described as acute, at least not without being supplemented by further context. Alternatively, or additionally, another learned rule can be one that does not allow a systemic disease to be described as a non-invasive disease. In some implementations, criterion can include temporal limitations such as when a disease can be characterized differently from a point in time that the disease was originally characterized. For example, a particular report that is summarized as describing a clinical concept, such as a patient experiencing acute abdominal pain, can be relied upon as long as no other report generated prior to the particular report includes content characterizing the same patient suffering from Crohn's disease. In other words, a diagnosis of Crohn's disease may be the result of medical analysis that may have occurred subsequent to the particular report (e.g., the report describing acute abdominal pain), and therefore, if Crohn's disease is not cited in subsequent reports, incorrect clinical pathways may be selected by a medical professional who is not aware of the Crohn's disease diagnosis.

The method 300 further include an operation 308 of identifying, based on the first report characterizing the clinical feature, a second report that corresponds to the one or more patients and is associated with the clinical feature. The second report can be identified in order to assist the user in resolving any conflicts and/or inaccuracies that may not be immediately apparent, if the user were to rely on the first report. In some implementations, multiple different reports can be stored in association with the one or more patients, and when the user identifies the first report, the multiple different reports can be searched based on one or more clinical features being characterized by the first report. For example, the first report can characterize a clinical feature such as acute abdominal pain, and the multiple different reports can be searched in order to identify any other content associated with acute abdominal pain. As a result of the search, the second report can be identified, at least based on the second report for example, including the term Crohn's disease. Alternatively, or additionally, multiple different summaries of multiple different reports can be searched for terms related to the clinical feature. For example, although the second report identifies Crohn's disease, summary data corresponding to the second report can include other terms such as abdominal, pain, chronic, autoimmune, and/or any other terms that can characterize Crohn's disease. Therefore, when the multiple different summaries of the multiple different reports are being searched using the terms “acute,” “abdominal,” and “pain,” the second report can be identified at least based on the summary of the second report including the aforementioned other terms.

The method 300 can proceed from operation 310 via continuation element “B” to operation 314 of the method 312, as illustrated in FIG. 3A and FIG. 3B. The operation 314 can include determining whether the first source conflicts with the second source. In some implementations, the operation 314 can include determining whether a criterion of a clinical concept described by the first report is satisfied or otherwise not invalidated by content of the second report. For example, if the first report includes content describing acute abdominal pain and the second report includes content describing Crohn's disease, and if the first report was generated subsequent to the second report, the first report can be considered to conflict with the second report, and therefore not satisfy the criterion corresponding to the clinical concept “Crohn's disease.” In other words, because the first report came after the second report and the first report did not explicitly describe Crohn's disease, the first report can be considered to conflict with the second report. If no conflict between the first report and the second report is determined, the method 312 can proceed from the operation 314 to the operation 318. However, if the first report is determined to conflict with the second report, the method 312 can proceed from the operation 314 to the operation 316.

The operation 316 can include generating supplemental data that characterizes one or more conflicts between content of the first report and other content of the second report. In some implementations, the supplemental data can characterize a context in which the first report and/or the second report is regenerated. Alternatively, or additionally, the supplemental data can characterize a criterion that was not satisfied with respect to a particular clinical concept described by the first report and/or the second report. For example, in the aforementioned scenario, if the second report explicitly describes Crohn's disease but the first report only describes acute abdominal pain, the supplemental data that is generated can include natural language content describing criterion corresponding to the clinical concept “Crohn's disease.” Therefore, in response to the user initializing the carryover and/or other transfer of data from the first report to the targeted report, the supplemental data can be included in the carryover process. In some implementations, the natural language content of the supplemental data can be incorporated into the targeted report based on the first report conflicting with the second report. Alternatively, or additionally, natural language content of the supplemental data can be supplanted into the first report in the same field, and/or a different field, to which the “acute abdominal pain” description from the first report is being copied.

The method 312 can proceed from the operation 316 to the operation 318. In some implementations, the method 300 can proceed from the operation 310 to the operation 318 of the method 312. The operation 318 causing the user interface, or another user interface of another computing device, to graphically render the first data and, optionally, the supplemental data. The operation 318 can include causing the user interface, or another user interface of another computing device, to graphically render the transfer of the first data from the first report to the target source. Optionally, the operation 318 can include graphically rendering the transfer of the supplemental data to the target source. In other words, if the first data from the first report is the description of “acute abdominal pain,” then that description can be incorporated into a field of the target source. Furthermore, in some instances when there is a conflict between the first report in the second report, supplemental data that is based on a criterion of a clinical concept not being satisfied, can also be incorporated into the field, or a different field, of the target report. In this way, any subsequent medical professional, and/or patient, relying on the target report can be on notice that there is at least one conflict between data of certain reports that have been generated for a patient over time. It should be noted that although medical data has been referenced herein, any other type of data can be processed via any of the devices, operations, methods, engines, and/or any other apparatus or module discussed herein.

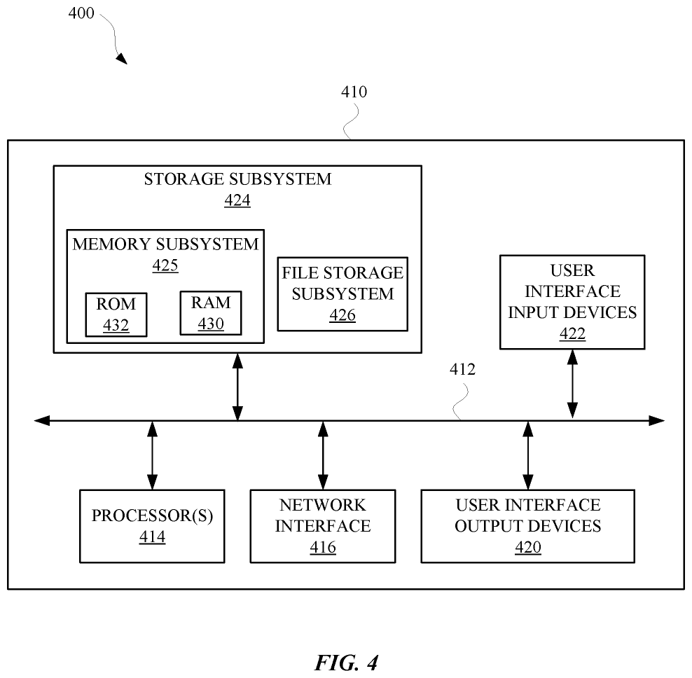

FIG. 4 is a block diagram of an example computer system 410. Computer system 410 typically includes at least one processor 414 which communicates with a number of peripheral devices via bus subsystem 412. These peripheral devices may include a storage subsystem 424, including, for example, a memory 425 and a file storage subsystem 426, user interface output devices 420, user interface input devices 422, and a network interface subsystem 416. The input and output devices allow user interaction with computer system 410. Network interface subsystem 416 provides an interface to outside networks and is coupled to corresponding interface devices in other computer systems.

User interface input devices 422 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 410 or onto a communication network.

User interface output devices 420 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 410 to the user or to another machine or computer system.

Storage subsystem 424 stores programming and data constructs that provide the functionality of some or all of the modules described herein. For example, the storage subsystem 424 may include the logic to perform selected aspects of method 300, method 312, and/or to implement one or more of computing device 110, computing device 122, computing device 210, client device 220, and/or any other device, operation, application, and/or engine discussed herein.

These software modules are generally executed by processor 414 alone or in combination with other processors. Memory 425 used in the storage subsystem 424 can include a number of memories including a main random access memory (RAM) 430 for storage of instructions and data during program execution and a read only memory (ROM) 432 in which fixed instructions are stored. A file storage subsystem 426 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 426 in the storage subsystem 424, or in other machines accessible by the processor(s) 414.

Bus subsystem 412 provides a mechanism for letting the various components and subsystems of computer system 410 communicate with each other as intended. Although bus subsystem 412 is shown schematically as a single bus, alternative implementations of the bus subsystem may use multiple busses.

Computer system 410 can be of varying types including a workstation, server, computing cluster, blade server, server farm, or any other data processing system or computing device. Due to the ever-changing nature of computers and networks, the description of computer system 410 depicted in FIG. 4 is intended only as a specific example for purposes of illustrating some implementations. Many other configurations of computer system 410 are possible having more or fewer components than the computer system depicted in FIG. 4.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope

What is claimed is:

1. A method implemented by one or more processors, the method comprising:
processing, via the one or more processors and in response to an input received at a user interface of a computing device, input data that indicates a user has initialized a transfer of first data from a first source to a target source;
identifying, based on processing the input data, (i) first data characterizing a clinical feature of one or more patients, and (ii) a criterion corresponding to the clinical feature, wherein the first data is based on natural language content of the first source;
identifying, based on identifying the first data, a second source that is different from the first source and associated with the clinical feature,
wherein the second source includes other natural language content that is associated with the clinical feature;
determining whether the first data and the second data satisfy the criterion corresponding to the clinical feature; and
when the first data and the second data are determined to not satisfy the criterion corresponding to the clinical feature:
generating supplement data that indicates the first data and the second data do not satisfy the criterion corresponding to the clinical feature, and
causing, at least in response to the input received at the user interface of the computing device, the user interface, or another user interface of another computing, to graphically render the first data and the supplemental data.

2. The method of claim 1, wherein the target source is an application file generated by the user via the user interface of the computing device, and the application file includes a field to which the first data is to be incorporated.

3. The method of claim 1, wherein causing the user interface, or the other user interface of the other computing device, to render the first data and the supplemental data includes:
causing the user interface, or the other user interface of the other computing device, to simultaneously render the first data and the supplemental data.

4. The method of claim 1, wherein determining whether the first data and the second data satisfy the criterion includes determining whether a temporal aspect of the first data and another temporal aspect of the second data satisfy the criterion.

5. The method of claim 1, wherein the temporal aspect characterizes a time at which the first data was generated or last modified, and the other temporal aspect characterizes another time at which the second data was generated or last modified.

6. The method of claim 1,
wherein the first data is provided by the first source, and
wherein determining whether the first data and the second data satisfy the criterion includes determining whether the second data characterizes conflicting content relative to the clinical feature characterized by the first data.

7. The method of claim 6, wherein the criterion is generated based on one or more trained machined learning models that are trained according to patient medical records and/or medical reference data.

8. A non-transitory computer-readable medium storing instructions that, when executed by one or more processors, cause the one or more processors to perform operations that include:
processing, via the one or more processors and in response to an input received at a user interface of a computing device, input data that indicates a user has initialized a transfer of first data from a first source to a target source;
identifying, based on processing the input data, (i) first data characterizing a clinical feature of one or more patients, and (ii) a criterion corresponding to the clinical feature, wherein the first data is based on natural language content of the first source;
determining whether another source of data is associated with the one or more patients and the clinical feature;
when no other source of data is determined to be associated with the one or more patients and the clinical feature:
causing, at least in response to the input received at the user interface of the computing device, the user interface, or another user interface of another computing, to graphically render the first data; and
when a second source of data is determined to be associated with the one or more patients and the clinical feature:
determining, based on the criterion that corresponds to the clinical feature, whether the first data and the second data satisfy the criterion corresponding to the clinical feature, and
when the first data and the second data are determined to not satisfy the criterion corresponding to the clinical feature:
generating supplement data that indicates the first data and the second data do not satisfy the criterion corresponding to the clinical feature, and
causing, at least in response to the input received at the user interface of the computing device, the user interface, or another user interface of another computing, to graphically render the first data and the supplemental data.

9. The non-transitory computer-readable medium of claim 8, wherein the target source is an application file generated by the user via the user interface of the computing device, and the application file includes a field to which the first data is to be incorporated.

10. The non-transitory computer-readable medium of claim 8, wherein causing the user interface, or the other user interface of the other computing device, to render the first data and the supplemental data includes:
causing the user interface, or the other user interface of the other computing device, to simultaneously render the first data and the supplemental data.

11. The non-transitory computer-readable medium of claim 8, wherein determining whether the first data and the second data satisfy the criterion includes determining whether a temporal aspect of the first data and another temporal aspect of the second data satisfy the criterion.

12. The non-transitory computer-readable medium of claim 8, wherein the temporal aspect characterizes a time at which the first data was generated or last modified, and the other temporal aspect characterizes another time at which the second data was generated or last modified.

13. The non-transitory computer-readable medium of claim 8,
wherein the first data is provided by the first source, and
wherein determining whether the first data and the second data satisfy the criterion includes determining whether the second data characterizes conflicting content relative to the clinical feature characterized by the first data.

14. The non-transitory computer-readable medium of claim 13, wherein the criterion is generated based on one or more trained machined learning models that are trained according to patient medical records and/or medical reference data.

15. A system, comprising:
one or more processors; and
memory storing instructions that, when executed by the one or more processors, cause the one or more processors to perform operations that include:
processing, via the one or more processors and in response to an input received at a user interface of a computing device, input data that indicates a user has initialized a transfer of first data from a first source to a target source;
identifying, based on processing the input data, (i) first data characterizing a clinical feature of one or more patients, and (ii) a criterion corresponding to the clinical feature, wherein the first data is based on natural language content of the first source;
identifying, based on identifying clinical feature of the one or more patients, a second source that is different from the first source and associated with the clinical feature,
wherein the second source includes other natural language content that is associated with the clinical feature and was generated at a different time than the natural language content of the first source was generated;
determining, based on the criterion that corresponds to the clinical feature, whether the first data and the second data satisfy the criterion corresponding to the clinical feature; and
when the first data and the second data are determined to not satisfy the criterion corresponding to the clinical feature:
generating supplement data that indicates the first data and the second data do not satisfy the criterion corresponding to the clinical feature, and
causing, at least in response to the input received at the user interface of the computing device, the user interface, or another user interface of another computing, to graphically render the first data and the supplemental data.

16. The system of claim 15, wherein the target source is an application file generated by the user via the user interface of the computing device, and the application file includes a field to which the target data is to be provided.

17. The system of claim 15, wherein causing the user interface, or the other user interface of the other computing device, to render the first data and the supplemental data includes:
causing the user interface, or the other user interface of the other computing device, to simultaneously render the first data and the supplemental data.

18. The system of claim 15, wherein determining whether the first data and the second data satisfy the criterion includes determining whether a temporal aspect of the first data and another temporal aspect of the second data satisfy the criterion.

19. The system of claim 15, wherein the temporal aspect characterizes a time at which the first data was generated or last modified, and the other temporal aspect characterizes another time at which the second data was generated or last modified.

20. The system of claim 15, wherein the criterion is generated based on one or more trained machined learning models that are trained according to patient medical records and/or medical reference data.
